(19) United States
(12) Patent Application Publication
(10) Pub. No.: US 2008/0255465 A1
(43) Pub. Date: Oct. 16, 2008

(54) Heart-function information display including color-range scalogram content

(75) Inventor: Alex T. Nelson, Portland, OR (US)

Correspondence Address: ROBERT D. VARITZ, P.C., 4915 SE 33RD PLACE, PORTLAND, OR, 97202, US

(73) Assignee: Inovise Medical, Inc.

(21) Appl. No.: 12/005,555

(22) Filed: Dec. 26, 2007

Related U.S. Application Data
(60) Provisional application No. 60/923,580, filed on Apr. 16, 2007.

(51) Int. Cl. A61B 5/044

(52) U.S. Cl. 600/523

(57) ABSTRACT

A method employing electronic processing for presenting a heart-function informational display including the steps of (a) gathering, over a selected time interval, a person's ECG and heart-sound signals, (b) electronically sampling and digitizing such gathered signals, and (c) electronically processing those signals to create a scalogram wherein colors which lie within a selected range of colors are employed to indicate energy-based content reflected in the signals.

### CROSS REFERENCE TO RELATED APPLICATION

This application claims filing-date priority to the filing date, Apr. 16, 2007, of currently co-pending U.S. Provisional Patent Application Ser. No. 60/923,580 covering an invention entitled “Scalogram”. The entire disclosure content of that provisional application is hereby incorporated herein by reference.

### BACKGROUND AND SUMMARY OF THE INVENTION

The present invention pertains to a methodology for acquiring and presenting graphically interrelated heart-function ECG and sound information. In particular, it pertains to such methodology which produces a highly intuitive graphical display that includes scalogram content, wherein colors drawn from a continuity range of colors are employed to indicate energy levels that are associated with each time-frequency location presented in the scalogram content.

The invention specifically addresses many of the interpretation difficulties so often associated with efforts to extract important meaningful information contained in a standard plot of heart-function waveform information as a function of time.

In the practice of the proposed present-invention methodology, directly acquired input information includes (a) digitally sampled heart-sound data, and (b) digitally sampled ECG data. An appropriate sampling rate for sound data is 1000-samples-per-second, and an appropriate sampling rate for ECG data is 500-samples-per-second.

Sampled heart-sound and ECG data are collectively processed utilizing a conventional algorithmic approach for detecting various heart-function behaviors including, but not necessarily limited to, Q-onset, the S₁, S₂, S₃ and S₄ heart sounds, murmur, the mitral component of S₁, the aortic component of S₂, the tricuspid component of S₁, and the pulmonic component of S₂.

These algorithmically detected heart-function features, along with appropriately filtered streams of heart-sound and ECG data, per se, are effectively wavelet transformed to generate plotable, graphical scalogram content. A user-selectable, continuous range of colors, and appropriately selected energy-level thresholds, are applied to the generated scalogram content to enable the ultimate outputting of plotable (including display-screen-presentable), intuitively informative scalogram information wherein, as mentioned above, different colors represent different, respective energy levels at each time-frequency location in the scalogram graphical plot.

Preferably, output scalogram information is suitably marked to indicate the approximate locations of one or more of the several kinds of specific heart-function behaviors mentioned above herein. Preferably also, output scalogram information is presented on, and along, a common time base with related, sampled heart-sound and ECG information.

These and other important features of the present invention will shortly become more fully apparent as the description thereof which now follows is read in conjunction with the accompanying drawings.

### DESCRIPTION OF THE DRAWINGS

FIG. 1 is a block/schematic drawing which illustrates the preferred and best-mode methodology of the present invention, as well as a system for implementing that methodology.

FIG. 2 illustrates, on a generally common time scale, various forms of graphical output information, including color-gradient scalogram information, created in accordance with implementation and practice of the invention pictured in FIG. 1.

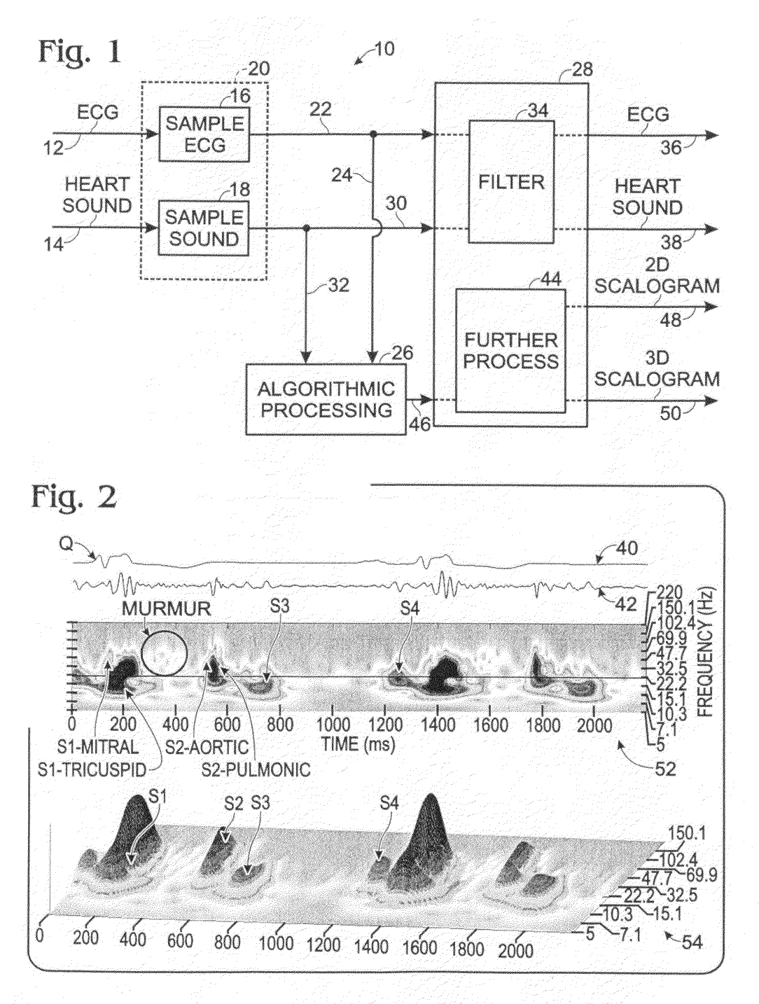

### DETAILED DESCRIPTION OF THE INVENTION

Turning now to the drawings, indicated generally at 10 are both the methodology, and a system for implementing that methodology, of a preferred and best-mode form of the present invention.

Analogue ECG and heart-sound data, over a user-selected time interval, are appropriately and conventionally collected from a person, and are fed over conductive pathways 12, 14, respectively, to digital sampling structures 16, 18, respectively, which reside in a digital sampling block 20. ECG sampling in structure 16 is performed herein preferably (though not necessarily) at the rate of 500-samples-per-second, and heart-sound sampling in structure 18 is performed (though not necessarily) at the rate of 1000-samples-per-second.

Digitally sampled ECG data is supplied by conductive pathways 22, 24 to an algorithmic processing block 26, and to a final signal-processing block 28 as illustrated. Digitally sampled heart-sound data is supplied by conductive pathways 30, 32 to processing blocks 26, 28.

Within processing block 28, ECG and heart-sound signal data are appropriately and conventionally filtered by a block shown at 34, with filtered ECG and heart-sound data being provided as plotable/displayable time-base data on output conductive pathways 36, 38, respectively. This output data is effective to create the time-based graphical waveforms shown in FIG. 2 at 40 for ECG data, and at 42 for heart-sound data.

Also within processing block 28, what is referred to herein as a further processing block 44 cooperates with algorithmic processing block 26, via a feed of information from the latter to the former over a conductive pathway 46, to generate plotable/displayable, time-based, wavelet-transformed scalogram content output information in either one, or both if desired, of two different ways on conductive pathways 48, 50. Scalogram content provided on pathway 48 is effective to create a scalogram display such as that shown at 52 in FIG. 2. Scalogram content provided on pathway 50 is effective to create a scalogram display such as that shown at 54 in FIG. 2.

Within processing block 26, any one of a number of user-selectable, conventional signal-processing algorithms is employed to detect and locate specific heart-function behaviors of diagnostic interest, such as the several specific such behaviors mentioned earlier herein.

In relation to the cooperative wavelet-transforming activities associated with blocks 26, 44, calculations are suitably performed to determine the energy content at each relevant time-frequency location in the resulting scalogram content information. Such determined energy content will present a range of distributed energy-content values, each associated with a particular time-frequency location. In regard to this energy-content range, upper and lower power-percentile “threshold” values are established, such as a 2%-value for the lower value, and a 98%-value for the upper value. With these values established, a continuous range of colors is chosen to be associated with the determined energy range. Energy-content values below the 2%-marker will all be associated with a single color located at one end of the chosen range of colors. Energy-content values above the 98%-marker will all be associated with another single color located at the other end of the chosen range of colors. Energy-content values which lie within the relevant range will be assigned respectively different colors that are distributed within the color range.

All of the above-described signal processing which is performed in the practice of the invention now being described, which processing will typically be carried out by a suitably programmed digital computer, results in the outputting of time-based waveforms 40, 42 as shown in FIG. 2, as well as the outputting of scalograms 52, 54, also as shown in FIG. 2.

Regarding the scalogram output information, scalogram 40 may effectively be thought of as being a 2-dimensional (2D) graphical representation of heart activity, with time being presented along the illustrated horizontal axis, frequency being presented along the vertical axis, and energy content at each time-frequency location being represented by a particular color within a gradation of colors lying in the established color range. Associated with scalogram 40 are verbal indicators of the approximate locations of graphically presented information relating to certain specific heart-function behaviors such as those mentioned earlier herein.

Scalogram 54 may effectively be thought of as being a virtual, 3-dimensional (3D) graphical representation of heart activity, with time being presented along the illustrated horizontal axis, energy-content level for each time-frequency location being presented in color along the vertical axis, and frequency being presented along the pictured oblique axis. Verbal indicators like those employed in scalogram 52 are also seen in scalogram 54.

Further with respect to what is shown in FIG. 2, it should be understood that the preferred form of the present invention is one which makes available, as included output information, both the 2D and the 3D scalograms shown at 52 and 54 in FIG. 2. The invention may also, of course, be differently configured, such as to furnish a one-only type of color-gradient scalogram. The invention may also be configured readily to identify and mark in a presented scalogram the approximate time-frequency locations of a greater or lesser number of specific heart-function behaviors such as those specifically discussed and illustrated herein.

Thus, a unique methodology for presenting a highly intuitive heart-function informational display has been described and illustrated herein. This methodology, in broadly stated terms, includes the steps of (a) gathering, over a selected time interval, a person's ECG and heart-sound signals, (b) electronically sampling and digitizing such gathered signals, and (c) electronically processing those signals to create a scalogram wherein colors which lie within a selected range of colors are employed to indicate energy-based content reflected in the signals. From this methodologic practice, the information presentation which is ultimately created is clearly highly intuitive in nature It is made especially so because of the production and use, in the resulting presentation, of one, or several, scalogram display(s) that incorporate, point-by-point, time-and-frequency, energy-content information illustrated in the form of different colors lying within a selected color range.

Those skilled in the relevant art will recognize, of course, that variations and modifications may be made in the methodologic character of the invention in ways that lie within the spirit and scope of the invention.

I claim:

1. A method employing electronic processing for presenting a heart-function informational display comprising
gathering, over a selected time interval, a person's ECG and heart-sound signals,
electronically sampling and digitizing such gathered signals, and
electronically processing the sampled and digitized signals to create a visually presentable wavelet scalogram thereof in the form of a plural-axis time-and-frequency-content graphical energy display, with energy content present at each time-frequency location in the display represented by a predetermined, associated color which lies within a selected range of colors.

2. The method of claim 1 which further comprises, prior to said creating, electronically processing the sampled and digitized signals to detect selected heart-function behaviors reflected therein.

3. The method of claim 1, wherein the created plural-axis display includes time and frequency axes.

4. The method of claim 2, wherein the created plural-axis display includes time and frequency axes.

5. The method of claim 1, wherein the created plural-axis display includes time, frequency and energy-level axes.

6. The method of claim 2, wherein the created plural-axis display includes time, frequency and energy-level axes.

7. A method employing electronic processing for presenting heart-function information comprising
gathering over a selected time interval a person's ECG and heart-sound signals, and
electronically processing those signals to create a visually presentable graphic display which, in relation to at least a portion of the mentioned time interval, and utilizing colors which lie within a selected range of colors, simultaneously, utilizing plural display axes, time frequency and energy content of plural locations distributed within the gathered signals.

8. The method of claim 7, wherein said electronic processing includes signal sampling, digitizing and wavelet transforming.

9. The method of claim 7, wherein said creating involves producing a scalogram display.

10. A method employing electronic processing for presenting a heart-function informational display comprising
gathering over a selected time interval a person's ECG and heart-sound signals,
electronically sampling and digitizing such gathered signals, and
electronically processing those signals to create a scalogram wherein colors which lie within a selected range of colors are employed to indicate energy-based content reflected in the signals.
